(19) United States
(12) Patent Application Publication
(10) Pub. No.: US 2025/0354144 A1
(43) Pub. Date: Nov. 20, 2025

(54) TARGETING MUC1-C WITH A NOVEL ANTISENSE OLIGONUCLEOTIDE FOR THE TREATMENT OF CANCER

(71) Applicant: Dana-Farber Cancer Institute, Inc., Boston, MA (US)

(72) Inventor: Donald W. Kufe, Boston, MA (US)

(73) Assignee: Dana-Farber Cancer Institute, Inc., Boston, MA (US)

(21) Appl. No.: 18/871,966

(22) PCT Filed: Jun. 7, 2023
(86) PCT No.: PCT/US2023/068059

§ 371 Date: Dec. 5, 2024

Related U.S. Application Data
(60) Provisional application No. 63/349,927, filed on Jun. 7, 2022.

(51) Int. Cl. C12N 15/113

(52) U.S. Cl. CPC C12N 15/113; C12N 2310/11; C12N 2310/531

(57) ABSTRACT

The present disclosure relates to novel antisense oligonucleotides targeting MUC1-C, for the treatment of cancers, including but not limited to neuroendocrine cancers, including Merkel cell carcinoma (MCC); small cell lung cancer, breast cancer, colorectal cancer or prostate cancer.

### CROSS REFERENCE TO RELATED APPLICATIONS

The present disclosure claims the benefit of and priority to U.S. Provisional Application No. 63/349,927, filed Jun. 7, 2022, the contents of which are incorporated by reference in its entirety.

### STATEMENT REGARDING SEQUENCE LISTING

The Sequence Listing associated with this application is provided in XML format in lieu of paper copy, and is hereby incorporated by reference into the specification. The name of the XML file containing the Sequence Listing is “2023-06-06_91016-388437_Sequence Listing ST26”. The XML file is 6.76 KB, was created on Jun. 6, 2023, and is being submitted electronically via Patent Center, concurrent with the filing of this specification.

### FIELD

The present disclosure relates to novel antisense oligonucleotides targeting the MUC1-C gene, for the treatment of cancer, including but not limited to neuroendocrine cancers, including Merkel cell carcinoma (MCC), colorectal cancer, breast cancer or prostate cancer.

### BACKGROUND OF THE INVENTION

Blood and bone marrow cancers, such as leukemia, lymphoma, and myeloma make up almost 10% of new cancer cases that will be diagnosed in the U.S. in 2022. Further, each year, the incidence of neuroendocrine cancers, including Merkel cell carcinoma; breast cancer, lung cancer, non-small cell lung cancer, small cell lung cancer, colorectal cancer, pancreatic cancer, or prostate cancer, is about 2.5-5 per 100,000 people with a significant increase as detection methodologies improve. Although some treatments are available, they demonstrate limited efficacy or availability. Therefore, additional treatments for blood and bone marrow cancers as well as neuroendocrine cancers are desired.

### SUMMARY OF THE INVENTION

In one embodiment described herein is an antisense oligonucleotide of Formula (I): AZₘGYₙGXT, wherein Z is independently, in each occurrence, any nucleotide; Y is independently in each occurrence any nucleotide; X is A or G; m=3; and n=7; or a pharmaceutically acceptable salt thereof.

In one aspect, the antisense oligonucleotide comprising a length of 10-20 nucleotides. In another aspect, the antisense oligonucleotide is a nucleotide sequence of SEQ ID NOs: 3-6. In another aspect, the antisense oligonucleotide is optionally modified. In another aspect, the antisense oligonucleotides target comprises an mRNA transcript. In another aspect, the mRNA transcript is a MUC1-C mRNA transcript.

Another aspect described herein is a pharmaceutical composition comprising one or more of the antisense oligonucleotide described herein or a pharmaceutically acceptable salt. In another aspect, the composition further comprises a lipid nanoparticle.

Another aspect described herein is a method of reducing cell viability comprising contacting a MUC1-C expressing cancer cell comprising contacting the cancer cell with any of the antisense oligonucleotides or pharmaceutical compositions described herein. In another aspect, is a method of treating cancer comprising administering to a subject in need thereof, an effective amount of any of the compositions described herein. In one aspect, the cancer comprises neuroendocrine cancer, including but not limited to Merkel cell carcinoma. In another aspect, the cancer comprises, leukemia, lymphoma, or myeloma. In another aspect, the cancer comprises breast cancer, lung cancer, non-small cell lung cancer, small cell lung cancer, colorectal cancer, or prostate cancer. In another aspect, the cancer comprises pancreatic cancer. In one aspect, the leukemia is acute myeloid leukemia. In another aspect, the myeloma is multiple myeloma.

In one aspect, the composition may be administered intravenously or topically. In another aspect, the composition may be administered in combination with chemotherapeutic agents, targeted inhibitors, immunotherapies or immune checkpoint inhibitors. In another aspect, the immunotherapy is an anti-MUC1-c antibody. In another aspect described herein is a method of inhibiting survival of a cell, comprising contacting a cell that expresses MUC1-C with any of the antisense oligonucleotides or pharmaceutical compositions described herein.

Another embodiment described herein is a method of treating cancer comprising administering to a subject in need thereof, an effective amount of a pharmaceutical composition comprising an antisense oligonucleotide of Formula (II): GPZₘGAYATXGA wherein P is G or T; Z is independently in each occurrence any nucleotide; Y is G or C; X is A or T; and m=3; or a pharmaceutically acceptable salt thereof.

### BRIEF DESCRIPTION OF THE FIGURES

FIG. 1A: describes relative mRNA levels after the MUC1-C gene was silenced in MCC26 cells transfected with the antisense oligonucleotides described herein.

FIG. 1B: describes percent cell death after MUC1-C silenced MCC26 cells were transfected with the antisense oligonucleotides described herein.

FIG. 2: describes relative mRNA levels after MUC1-C silenced MCC26 cells were transfected with the antisense oligonucleotides described herein.

FIG. 3: describes immunoblot staining of lysates with an anti-MUC1-C antibody.

FIG. 4: describes relative mRNA levels after MUC1-C silenced in BT-549 breast cancer cells were transfected with the antisense oligonucleotides described herein.

FIG. 5: describes relative mRNA levels in MUC1-C silenced in DU-145 prostate cancer cells, demonstrating an up regulation in the relative mRNA of XIST.

FIG. 6: describes immunoblotted lysates of 145 prostate cancer cells demonstrating an upregulation of various pathway proteins.

### DETAILED DESCRIPTION OF THE INVENTION

The present disclosure relates to antisense oligonucleotides for the modulation MUC1-C, and methods of treating disease or conditions associated with their biological function, including the treatment of cancer. Also described herein are methods of treating blood and bone marrow cancers as well as neuroendocrine cancers, such as Merkel cell carcinoma; small cell lung cancer, breast cancer, colorectal cancer, or prostate cancer.

The MUC1 gene appeared in mammals to protect epithelia from inflammation and damage induced by exposure to the external environment and is overexpressed in some carcinomas as well as contributes to diverse hallmark traits in some cancer cells (Kufe, 2009, 2020, 2013).

Merkel cell carcinoma (MCC) is an aggressive and recalcitrant neuroendocrine cancer with no effective targeted therapies (Kufe, 2009, 2020). The instant disclosure shows that MUC1 is expressed in both MCPyV-positive MCC (MCCP) and MCPyV-negative MCC (MCCN) tumors. This disclosure shows that silencing MUC1-C in MCCP and MCCN cells suppresses expression of (i) MYCL, (ii) pluripotency factors, and (iii) neuroendocrine differentiation transcription factors (TFs). In addition, this disclosure shows that MUC1-C suppresses DNA replicative stress, DNA damage and apoptosis. Further shown in this disclosure, targeting MUC1-C genetically and pharmacologically inhibits MCC cell self-renewal capacity and tumorigenicity.

Therapies targeting MUC1-C for the treatment of various cancers are disclosed herein, including leukemia, myeloma, lymphoma, and neuroendocrine cancers such as Merkel cell carcinoma; small cell lung cancer, breast cancer, colorectal cancer, or prostate cancer.

Unless otherwise defined, all technical and scientific terms used herein have the same meaning as commonly understood by one of ordinary skill in the art. In case of conflict, the present document, including definitions, will control Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

As used herein, the articles “a,” “an,” and “the” are used herein to refer to one or to more than one (i.e., to at least one) of the grammatical object of the article. By way of example, “an element” can mean one element or more than one element.

As used herein, the term “about” or “approximately” refers to a quantity, level, value, number, frequency, percentage, dimension, size, amount, weight or length that varies by as much as 10, 9, 8, 7, 6, 5, 4, 3, 2 or 1% to a reference quantity, level, value, number, frequency, percentage, dimension, size, amount, weight or length. In some embodiments, the terms “about” or “approximately” when preceding a numerical value indicates the value plus or minus a range of 10%, 5%, or 1%.

As used herein, “an effective amount” refers to an amount that causes relief of symptoms of a disorder or disease as noted through clinical testing and evaluation, patient observation, and/or the like. An “effective amount” may further designate a dose that causes a detectable change in biological or chemical activity. The detectable changes may be detected and/or further quantified by one skilled in the art for the relevant mechanism or process. Moreover, an “effective amount” may designate an amount that maintains a desired physiological state, i.e., reduces or prevents significant decline and/or promotes improvement in the condition of interest. An “effective amount” may further refer to a “therapeutically effective amount”.

As used herein, the term “antisense oligonucleotide” means a plurality of linked nucleosides, at least a portion of which, is complementary to a target nucleic acid to which it is capable of hybridizing, resulting in at least one antisense activity. In one aspect described herein, oligonucleotides comprise one or more of deoxyribonucleosides (DNA) and/or ribonucleosides (RNA). As used herein a “nucleotide” means a nucleoside further comprising a phosphate linking group. The nucleotides described herein may be found in both DNA and RNA, and may be referred to by their full name, or single letter abbreviation, all interchangeably.

The antisense oligonucleotides may be further modified in ways that either improve the delivery of the molecule to the target cells or tissues, or improve some aspect of the antisense oligonucleotide itself, such as stability. The present disclosure thus contemplates such modifications, including those known in the art, and that are suitable for the intended purpose, (Roberts, 2020). Such modifications include modification by the addition of specific groups or moieties to the antisense oligonucleotide, or chemical modification of the antisense oligonucleotide itself. Thus, one aspect described herein, is an antisense oligonucleotide that may optionally include one or more additional features, such as conjugate groups, terminal groups or targeting moieties, or may be chemically modified, or remain unmodified. The term “modification” as used herein means modification of the antisense oligonucleotide either by addition of various groups, moieties and linkages, or chemical modification of the antisense oligonucleotides.

The specific examples and types of modifications provided herein are representative of the modifications contemplated by the present disclosure. Thus the present disclosure contemplates all or any modifications known in the art, and may be made independently or in combination with others, and based on the specific parameters of the antisense oligonucleotides and their target nucleic acids, cells and tissues.

Modifications may include for example, a “conjugate group”. A conjugate group is a group of atoms that may be attached to an antisense oligonucleotide via a “conjugate linker”. “Terminal groups” are a chemical group or group of atoms covalently linked to a either terminus of an antisense oligonucleotide. Terminal groups include but are not limited to conjugate groups, capping groups, phosphate moieties, protecting groups, modified or unmodified nucleosides, and two or more nucleosides that are independently modified or unmodified. Described herein are representative conjugate groups, but any conjugate group known in the art may be suitable.

Conjugate groups may consist of one or more conjugate moiety and a conjugate linker, which links the conjugate moiety to the antisense oligonucleotide. Conjugate groups may be attached to either or both ends of an antisense oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Conjugate linkers include “linker nucleosides” that are nucleosides that link an antisense oligonucleotide to one or more conjugate moieties. The linker nucleoside is not considered part of the base antisense oligonucleotide, even if they are contiguous with the base antisense oligonucleotide itself. Conjugate linkers are known in the art, and include, for example, phosphodiester linkers, cleavable and non-cleavable linkers, phosphorothioate linkers, phosphonate linkers, nuclease-sensitive linkers, fluorescence-labeled nucleoside linkers, acid-labile linkers, disulfide linkers, and alkylamino linkers. See, for example, Hu et al., 2020; Roberts et al., 2020; Subramanian et al, 2015; and Reynold et al, 1996, each of which is incorporated by reference with regard to such background teaching.

Conjugate groups may also serve as a “targeting moiety” that bind to a specific cell or tissue type, and thus help in delivery of the antisense oligonucleotide to the target. Examples of such conjugate groups are known in the art, including but not limited to, lipids (cholesterol type molecules), peptides, antibodies and sugars.

A “Sugar moiety” may be either unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, substituted, or unsubstituted, and they may or may not have a stereoconfiguration other than β-D-ribosyl. Modified furanosyl sugar moieties include bicyclic sugars and non-bicyclic sugars.

Examples of sugar moieties include, but are not limited to, 4′ to 2′ bridging sugar substituents include, but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof. Other sugar moieties are known in the art. See for example Roberts et al., 2020, Elbashir et al., 2001, Geary et al., 2015 and Wan et al., 2016., each of which is incorporated by reference with regard to such background teaching.

Sugar conjugates may also include the use of carbohydrate molecules conjugated to the antisense oligonucleotide. Examples of such molecules include N-acetylgalactosamine (GalNAc), a sugar derivative of galactose, that is known to bind liver receptors with high affinity. (Bajan et al., 2020, incorporated by reference with regard to such background teaching). Conjugation of the GalNAc to the antisense oligonucleotide serves to guide the oligonucleotide to the target liver cells. The GalNAc is also an example of a “cleavable moiety” because it is subject to enzymatic degradation after it is has delivered the antisense oligonucleotide in the cell. Thus, “cleavable moiety” is a bond or group of bonds cleaved under specific physiological conditions.

Antibodies have long been used to direct pharmaceuticals to cells by specifically targeting cell surface receptors. The present disclosure thus contemplates the use of appropriate antibodies as conjugates in conjunction with the antisense oligonucleotides disclosed herein. In one aspect described herein, an anti-MUC1-C antibody may be a conjugate. In another aspect, the antibodies 3D1 and 7B8 (against the MUC1-C extracellular domain) are antibody conjugates to be conjugated to the antisense oligonucleotides described herein. However, any suitable antibody may be used. Antibody conjugates are known in the art, and antisense oligonucleotides have been conjugated with a variety of antibodies including, for example, CD44, EPHA2 and EGFR193. See for example, Song et al., 2005; Sugo et al., 2016; and Arnold et al., 2018, each of which is incorporated by reference with regard to such background teaching.

In addition to modification by the addition of groups, chemical modification may also be made to the antisense oligonucleotides described herein. As used herein “chemical modification” results in substitutions or alternations to the antisense oligonucleotide itself through chemical reaction. Chemical modifications include, but are not limited to, modifying sugar moieties, modifying internucleoside linkages, or modifying the nucleobases themselves. Modifications may occur independently of each other, and as suitable for the specific antisense oligonucleotide lengths and sequence motifs.

“Sugar modification” as used herein refers to a chemical modification of an existing sugar moiety within the antisense oligonucleotide, such as the addition of a substituent that does not form a bridge between two atoms of the sugar to form a second ring. These may further be referred to as “non-bicyclic modified sugar”. Both bicyclic and non-bicyclic modified sugars are known in the art. In some embodiments, the oxygen atom of the sugar moiety is replaced, e.g., with a sulfur, carbon or nitrogen atom. In certain such embodiments, such modified sugar moieties also comprise bridging and/or non-bridging substituents as described herein. “Sugar modifications” include modification at the 2′ position of the ribose sugar. “Sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an antisense oligonucleotide. Modified nucleosides comprising sugar surrogates can be incorporated into one or more positions within an antisense oligonucleotide and such antisense oligonucleotides are capable of hybridizing to complementary oligomeric compounds or nucleic acids. Sugar surrogates may also comprises rings having more than 5 atoms and more than one heteroatom. For example, nucleosides comprising morpholino sugar moieties and their use in oligonucleotides are known (Braasch et al., 2002, incorporated by reference with regard to such background teaching).

As used herein an “internucleoside linkage” refers to the covalent bond between nucleoside molecules within an oligonucleotide. Naturally occurring internucleoside linkages comprise a 3′ to 5′ phosphodiester bond. A “modified internucleoside linkage” thus refers to a non-naturally occurring linkage, such as for example, a non-phosphate linkage. Another example of an internucleoside linkage include “phosphorothioate linkages” or “PS linkages” in which the non-bridging oxygen atom of the inter-nucleotide phosphate group is replaced with a sulfur atom resulting in “backbone modifications”. PS linkages are known to be resistant to nuclease activity and may facilitate binding of the antisense oligonucleotide to proteins.

Representative internucleoside linkages having a chiral center include but are not limited to alkylphosphonates and phosphorothioates and representative phosphorus-containing internucleoside linkages include, but are not limited to, phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O—); and N,N′-dimethylhydrazine (—CH₂—N(CH₃)—N(CH₃)—).

“Nucleobase modification” includes the use of chemically modified nucleobases, such as the methylated bases 5-methylcytidine or 5′methyluridine, to enhance properties of the antisense oligonucleotides. Nucleobase modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds.

Examples of modified nucleobases include, but are not limited to 5-substituted pyrimidines, 6-azapyrimidines, alkyl or alkynyl substituted pyrimidines, alkyl substituted purines, and N-2, N-6 and 0-6 substituted purines, 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone. Nucleobase modifications are known in the art, including, for example, those described in Roberts et al, 2020; Bennett, 2019; and Shen et al., 2017, each of which is incorporated by reference with regard to such background teaching).

The chemical modifications described herein are examples of known modifications, however the present disclosure further contemplates any and all modifications known in the art, including alternative chemistries known in the art, see for example, Shen et al, 2017; Agrawal, 2021; and Watts, 2018, each of which is incorporated by reference with regard to such background teaching.

As used herein, the term “antisense activity” means any detectable and/or measurable change attributable to the hybridization of an antisense compound to its target nucleic acid. In one embodiment described herein, the antisense activity is modulation or alteration of the expression level of a target gene, DNA, RNA or protein, for example, antisense activity includes, but is not limited to a reduction, prevention or downregulation of MUC1-C gene expression, or MUC1-C mRNA expression or MUC1-C protein expression. Such modulation may be measured in ways that are routine in the art. In addition, effects on cancer cell proliferation or tumor growth are well known in the art.

As used herein, the term “complementary” in reference to oligonucleotides means the capacity of the oligonucleotide to hybridize to another oligonucleotide compound or region via established Watson-Crick nucleotide base pairing rules, resulting in hybridization. Some mismatches are tolerated, thus in one aspect, antisense oligonucleotides may be 70% complementary. In other aspects, antisense oligonucleotides may be 80% complementary. In some aspects, antisense oligonucleotides may be 90% complementary. In some aspects, antisense oligonucleotides may be 95% complementary. In yet other aspects, antisense oligonucleotides may be 99% complementary. In yet other aspects, antisense oligonucleotides may be 100% complementary.

“Hybridization” means the annealing of the antisense oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense oligonucleotide and a nucleic acid target. In certain embodiments, complementary nucleic acid molecules include, but are not limited to, an oligonucleotide and a nucleic acid target.

As used herein, the term “individual” and “subject” are often used interchangeably and refer to any human or domestic animal that may be treated with the methods disclosed herein. Suitable subjects (e.g., patients) include humans and domestic animals or pets (such as a cat or dog). Non-human primates and human patients are included. In one embodiment, subjects may include human patients that have been diagnosed with cancer, including but not limited to Merkel Cell Carcinoma (MCC); leukemia, myeloma, breast cancer, lung cancer (including non-small cell lung cancer (NSLC), and small cell lung cancer (SCLC), colorectal cancer, pancreatic cancer, multiple myeloma, acute myeloid leukemia, or prostate cancer. As used herein, the term “patient” refers to a subject that may receive a treatment of a disease or condition.

As used herein, “treatment”, “treat”, and “treating” refer to reversing, alleviating, mitigating, or slowing the progression of, or inhibiting the progress of, a disorder or disease or symptoms associated with such disorder or disease, and as described in more detail herein.

As used herein, “target nucleic acid” refers to the nucleic acid molecule or nucleic acid sequence to which an antisense oligonucleotide hybridizes to. In one aspect described herein, the target nucleic acid to which the antisense oligonucleotides bind is the MUC1-C gene. In another aspect, the target nucleic acid to which the antisense oligonucleotides bind to is DNA. In another aspect, the target nucleic acid to which the antisense oligonucleotides bind to is mRNA transcript. As used herein, “transcript” refers to an RNA molecule transcribed from DNA. Transcripts include, but are not limited mRNA, pre-mRNA, and partially processed RNA. “mRNA” means an RNA molecule that encodes a protein. In one aspect, the protein is MUC1-C.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The present disclosure describes novel antisense oligonucleotides for use in modulating the function of nucleic acid molecules encoding the MUC1-C protein, for the treatment of neuroendocrine cancers, including but not limited to Merkel cell carcinoma (MCC); breast cancer, colorectal cancer or prostate cancer. However, the antisense oligonucleotides contemplated herein may be used for the treatment of any cancer.

The MUC1-C coding sequence is 477 nucleotides in length and when expressed, is a transmembrane protein with a 58 amino acid long extracellular domain, a 28 amino acid long transmembrane domain and a 72 amino acid long cytoplasmic domain (Kufe, 2009). Without being bound by any theory, the cytoplasmic domain is believed to be involved in nuclear import and activation of various inflammatory pathways. (Kufe, 2009). SEQ ID NO: 1 describes the DNA sequence of MUC1-C and SEQ ID NO: 2 describes the amino acid sequence of MUC1-C with the extracellular domain italicized, the transmembrane domain underlined, and the cytoplasmic domain in lower caps. Target nucleic acid regions are shown in bold.

SEQ ID NO: 1: DNA sequence of MUC1-C
(SEQ ID NO: 1)
TCTGTGGTGGTACAATTGACTCTGGCCTTCCGAGAAGGTACCATC
AATGTCCACGACGTGGAGACACAGTTCAATCAGTATAAAACGGAA
GCAGCCTCTCGATATAACCTGACGATCTCAGACGTCAGCGTGAGT
GATGTGCCATTTCCTTTCTCTGCCCAGTCTGGGGCTGGGGTGCCA
GGCTGGGGCATCGCGCTGCTGGTGCTGGTCTGTGTTCTGGTTGCG
CTGGCCATTGTCTATCTCATTGCCTTGGCTGTCtgtcagtgccgc
cgaaagaactacgggcagctggacatctttccagcccgggatacc
taccatcctatgagcgagtaccccacctaccacacccatgggcgc
tatgtgccccctagcagtaccgatcgtagcccctatgagaaggtt
tctgcaggtaatggtggcagcagcctctcttacacaaacccagca
gtggcagccacttctgccaacttgtag.
SEQ ID NO: 2: Amino acid sequence of MUC1-C
(SEQ ID NO: 2)
SVVVQLTLAFREGTINVHDVETQFNQYKTEAASRYNLTISDVSV
SDVPFPFSAQSGAGVPGWGIALLVLVCVLVALAIVYLIALAVcq
crrknygqldifpardtyhpmseyptyhthgryvppsstdrspy
ekvsagnggsslsytnpavaatsanl*.

Antisense oligonucleotides may modulate the activity of the MUC1-C gene. Thus, one embodiment described herein is an antisense oligonucleotide specific for MUC1-C. In one aspect, the antisense oligonucleotides described herein are a complementary sequence to a specific portion of the MUC1-C DNA or mRNA transcript. Target sequences of the MUC1-C are noted Table 3 and include a target sequence in the extracellular domain, a target sequence in the transmembrane domain, and two sequences in the cytoplasmic domain:

TABLE 3
Target sequences of MUC1-C and antisense
oligonucleotides against them

| | | Antisense | ASO |
|---|---|---|---|
| ASO | MUC1-C target | oligo- | SEQ ID |
| Number | sequence* | nucleotide* | NO: |
| 38 | CATCAATGTCCACGAC | GTCGTGGACATTGATG | SEQ ID |
| | | | NO: 3 |
| 40 | TGTCTATCTCATTGCC | GGCAATGAGATAGACA | SEQ ID |
| | | | NO: 4 |
| 39 | ATCCTATGAGCGAGTA | TACTCGCTCATAGGAT | SEQ ID |
| | | | NO: 5 |
| 41 | ACCTACCACACCCATG | CATGGGTGTGGTAGGT | SEQ ID |
| | | | NO: 6 |

*All sequences are shown from their 3′ end to their 5′ end.

The MUC1 gene includes multiple exons with varying coding regions and is thus subject to alternative splicing and exon skipping. (Kumar, 2017). Thus, antisense oligonucleotides specific for target regions not subject to exon skipping may provide benefit. Activation of the MUC1-C cytoplasmic domain, is believed to be linked to the subversion of nuclear import and inflammation pathways. (Kufe, 2009). Thus, without being bound by any theory, it is believed that because of the specific role of the MUC1-C cytoplasmic domain in transforming healthy cells to cancerous cell, the antisense oligonucleotides of SEQ ID NO: 5 and 6, described herein, targeting the cytoplasmic domain of MUC1-C, also target splice variants that are subject to frequent exon skipping. SEQ ID NO: 4 targets the transmembrane domain and SEQ ID NO: 3 targets the extracellular domain.

Thus, in one embodiment described herein is an antisense oligonucleotide of Formula (I):

AZₘGYnGXT

wherein Z is any nucleotide; Y is any nucleotide; X is A or G; m=3 and n=7 or a pharmaceutically acceptable salt thereof.

In another embodiment describe herein, is an antisense oligonucleotide of Formula (II):

GPZₘGAYATXGA

wherein P is G or T, Z is any nucleotide; Y is G or C; X is A or T; and m=3 or pharmaceutically acceptable salt thereof.

In another aspect, the antisense oligonucleotides are single stranded DNA. In other aspects, the antisense oligonucleotides are single stranded RNA. The antisense oligonucleotides of the present disclosure may also be double stranded duplexes comprising a first antisense oligonucleotide having complementarity to the target nucleic acid, and a second antisense oligonucleotide having complementarity to the first antisense oligonucleotide.

In another aspect, the antisense oligonucleotides described herein are fully complementary to the target nucleic acid over the entire length of the oligonucleotide. In other aspect, the antisense oligonucleotides are 80% complementary to the target nucleic acid. In another aspect, the antisense oligonucleotides are 85% complementary to the target nucleic acid. In yet another aspect, the antisense oligonucleotides are 90% complementary to the target nucleic acid. In yet another aspect, the antisense oligonucleotides are 95% complementary to the target nucleic acid. In yet another aspect, the antisense oligonucleotides are 99% complementary to the target nucleic acid. In another aspect, the antisense oligonucleotides are 100% complementary to the target nucleic acid.

One advantage of antisense oligonucleotides is their size. Because these molecules are anywhere from 10-20 nucleobases long, they may be highly water soluble, stable, with short half-lives, they may undergo rapid endocytic uptake (endocytosis) by cells, all of which make them ideal for administration in saline solutions and for minimizing off target effects (Geary, 2015). Thus, in one aspect described herein, the antisense oligonucleotides are 6-20 nucleobases long. In another aspect, the antisense oligonucleotide are 10-20 nucleobases long. In yet another aspect, the antisense oligonucleotides are 10-15 nucleobases long. In one aspect, the antisense oligonucleotides are 16 nucleobases long.

These short pieces of DNA or RNA are capable of hybridizing to a target nucleic acid, resulting in at least one antisense activity. The diverse chemistries of these short oligonucleotides may be used to modulate expression through a variety of mechanisms, including but not limited to, endogenous RNase-H activity that recognizes DNA-DNA or DNA-RNA substrates for enzymatic degradation. Alternatively, the antisense oligonucleotides may sterically block RNA-RNA or RNA-protein interactions, or influence splicing decisions. (Roberts, 2020).

Thus, in certain aspects, hybridization of antisense oligonucleotides described herein to a target nucleic acid may or may not result in recruitment of a protein that cleaves the target nucleic acid. In other aspects, hybridization of the antisense oligonucleotides to the target nucleic acid results in alteration of splicing of the target nucleic acid. In other aspects, hybridization of the antisense oligonucleotides to a target nucleic acid results in inhibition of a binding interaction between the target nucleic acid and a protein or other nucleic acid. In certain other aspects, hybridization of the antisense oligonucleotide to a target nucleic acid results in alteration of translation of the target nucleic acid.

In certain embodiments, antisense oligonucleotides specifically hybridize to one or more target nucleic acids based on established Watson-Crick base pairing rules. In a certain aspect, the antisense oligonucleotide has a nucleobase sequence comprising a region having sufficient complementarity to a target nucleic acid sequence to allow hybridization and result in an antisense activity including interfering with the normal expression or function of the target molecule to cause a loss of utility, while having insufficient complementarity to any non-target sequences under conditions in which specific hybridization is desired. Alternatively, targeting of non-coding regions of a nucleic acid may result in transcriptional or translational activation.

In another aspect, the antisense oligonucleotides described herein may optionally be modified. In another aspect, the antisense oligonucleotides described herein optionally include one or more conjugate groups. In another aspect described herein, the antisense oligonucleotides optionally include any conjugate group known in the art. In one aspect, the conjugate group comprises an antibody. In another aspect, the conjugate group comprises an anti-MUC1-C antibody. In another aspect, the antisense oligonucleotides described herein may optionally be chemically modified. In another aspect, the chemical modification may be any chemical modification known in the art. In one aspect, the antisense oligonucleotides described herein may optionally be modified by addition of conjugating or targeting groups and/or moieties, and/or may also be optionally, chemically modified. In other aspect, the antisense oligonucleotides described herein are unmodified.

The antisense oligonucleotides of the present disclosure may also be utilized to achieve the desired pharmacological effect by administration to a patient in need thereof in an appropriately formulated pharmaceutical composition. A patient, for the purpose of this disclosure, is a domestic animal or a human, in need of treatment for a particular condition or disease. Therefore, in one aspect of the present disclosure is a pharmaceutical composition comprising a therapeutically effective amount of the antisense oligonucleotides described herein, and a pharmaceutically acceptable carrier.

The term “composition” as used herein is intended to encompass a product comprising specific ingredients in specified amounts, as well as any product which results, directly or indirectly, from combination of the specified ingredients in the specified amounts. By “pharmaceutically acceptable” it is meant the carrier, diluent or excipient must be compatible with the other ingredients of the formulation, including the antisense oligonucleotide, and not deleterious to the recipient thereof. A “pharmaceutically acceptable carrier” is any carrier which is relatively non-toxic and innocuous to a patient at concentrations consistent with effective activity of the active ingredient so that any side effects ascribable to the carrier do not vitiate the beneficial effects of the active ingredient.

Appropriate pharmaceutical compositions comprising the antisense oligonucleotides are contemplated herein, and are based partly on the specific tissues, and cell types involved. Pharmaceutical compositions appropriate for the antisense oligonucleotides of the instant disclosure may be thus be formulated according to any means know in the art. (See for example: Remington's Pharmaceutical Sciences, 15th Edition, chapter 33; Gagliardi et al., 2021; or Hammond et al., 2021).

Lipid nanoparticle (LNP) encapsulation of the antisense oligonucleotides is believed to be an approach for protecting the antisense oligonucleotides from degradation and facilitating cellular delivery. LNP's thus provide an opportunity to formulate compositions comprising antisense oligonucleotides that are less susceptible to eliciting an immune response and subsequent clearance. This may be due to the components of the lipid particle itself. The present disclosure thus contemplates the use of lipid nanoparticles to encapsulate the antisense oligonucleotides described herein. Thus, in one aspect described herein, is a pharmaceutical composition comprising any of the antisense oligonucleotides described herein, a pharmaceutically acceptable salt thereof, and a lipid nanoparticle.

Alternatively, polymeric nanoparticles comprises macromolecules selected to achieve specific properties, and may be customized in accordance with the drug delivery requirements. These synthetic nanoparticles may thus be tailored as needed. Exemplary polymeric nanoparticle materials include, but are not limited to, micelles, dendrimers, cyclodextrins, and polymeric vesicles. Commonly used polymers may include poly(lactic-co-glycolic acid) (PLGA), polyvinyl alcohol (PVA), or chitosan (CH) (Begines et al., 2020). Any appropriate polymeric nanoparticle is contemplated herein. Another aspect as described herein, is a pharmaceutical composition comprising any of the antisense oligonucleotides described herein, a pharmaceutically acceptable salt thereof, and a polymeric nanoparticle.

The pharmaceutical compositions for the administration of the compounds of this disclosure may conveniently be presented in unit dosage form and may be prepared by any of the methods well known in the art of pharmacy. All methods include the step of bringing the active ingredient into association with a carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

The antisense oligonucleotides described herein may be administered with a pharmaceutically-acceptable carrier using any effective conventional dosage unit forms, including, for example, immediate and timed release preparations, injectable, parenterally, topically or the like. In some aspects, the antisense oligonucleotide is administered intravenously, intra-arterially, intra-tumorally, subcutaneously, topically or via intraperitoneal administration, or as local, regional, systemic, or continual administration.

The silencing of MUC1-C in MCC cells that results in suppression of the expression of pluripotency and differentiation factors, along with the role of MUC1 in suppressing DNA stress, damage and apoptosis, indicates MCC cells are dependent on MUC1-C for both growth and viability. Silencing of MUC1-C along with the role of MUC1 in suppressing DNA stress, damage and apoptosis indicates MCC cells are dependent on MUC1-C for both growth and viability. Thus, targeting MUC1-C genetically and pharmacologically inhibits MCC cell self-renewal capacity and tumorigenicity. Thus described herein are methods of inhibiting MUC1-C expression, methods of inhibiting differentiation of a cell, methods of reducing cancer cell viability, methods of inhibiting the proliferation of cancer cells expressing MUC1-C, methods of increasing cell death, and methods of treating cancers. It is contemplated that any method or composition described herein may be implemented with respect to any other method of composition described herein.

Furthermore, MUC1-C expression may influence various gene expression patterns. For example, the XIST is a non-coding RNA on the X-chromosome of mammals and is an effector of X-chromosome inactivation during embryonic development. MUC1-C silencing may affect XIST expression, impacting the subsequent differentiation of the cell and ultimate cell fate. In one aspect, MUC1-C suppression may regulate expression of various genes and proteins, including but not limited to, WTAP, METTL3, METTL14, GAPDH, YTHDF2, IGF2BP1, CNOT1, TDP-43, ACTIN, CD44, Tubulin, or BMI1.

One aspect of the present disclosure is method of inhibiting the expression of MUC1-C, comprising contacting a cell that expresses MUC1-C with any of the antisense oligonucleotides or pharmaceutical compositions described herein; hybridizing the antisense oligonucleotides with their target sequence; and inhibiting MUC1-C expression.

Another aspect described herein is a method of inhibiting differentiation of a cell, comprising contacting a cell that expresses MUC1-C with any of the antisense oligonucleotides or pharmaceutical compositions described herein; hybridizing the antisense oligonucleotides with their target sequence; inhibiting MUC1-C expression.

Cell viability is impacted by the silencing of MUC1-C, where the term “cell viability” refers to the proportion of live, healthy cells in a population. Cell viability may be calculated by means known in the art, including calculating the ratio of total live/total cells in a population. Thus, another embodiment described herein is a method of reducing cancer cell viability, comprising: contacting a cell that expresses MUC1-C with any of the antisense oligonucleotides or pharmaceutical compositions described herein; hybridizing the antisense oligonucleotides with their target sequence.

Another aspect described herein, is a method of inhibiting the proliferation of cells expressing MUC1-C, comprising contacting a cell that expresses MUC1-C with any of the antisense oligonucleotides or pharmaceutical compositions described herein; hybridizing the antisense oligonucleotides with their target sequence.

Another aspect described herein, is a method of increasing cell death in a cell expressing MUC1-C, comprising contacting a cell that expresses MUC1-C with any of the antisense oligonucleotides or pharmaceutical compositions described herein; hybridizing the antisense oligonucleotides with their target sequence.

Another aspect described herein, is a method of increasing cell death in a MUC-C expressing cell, comprising contacting the cancer cells with any of the antisense oligonucleotides or pharmaceutical compositions described herein.

The MUC1-expressing cell may be cancer stem cell, a tumor cell, such as a carcinoma cell, a leukemia cell or a myeloma cell, such as a prostate or breast carcinoma cell. The cell may also be a somatic cell, a non-cancerous cell or a benign cell. The tumor cell may be located in a living subject. The living subject may be a human subject. In one aspect, the cell is a neuroendocrine cancer. In another aspect the neuroendocrine cancer is Merkel cell carcinoma.

In yet another aspect is a method of treating cancer comprising administering to a subject in need thereof, an effective amount of a pharmaceutical composition comprising any of the antisense oligonucleotides described herein.

In one aspect the cancer comprises neuroendocrine cancer. In another aspect the cancer comprises Merkel cell carcinoma (MCC). In another aspect, the cancer comprises breast cancer, lung cancer (including non-small cell lung cancer, small cell lung cancer), In another aspect, the cancer comprises colorectal cancer, pancreatic cancer, multiple myeloma, acute myeloid leukemia. In another aspect, the cancer comprises prostate cancer. Other cancers are also contemplated herein.

In another aspect, the subject is a human or a patient. In another aspect, the effective amount is any amount required to demonstrate a therapeutic effect. The therapeutically effective dosage of the antisense oligonucleotides of the disclosure may readily be determined for treatment of each desired indication. The amount of the active ingredient (e.g., antisense oligonucleotides) to be administered in the treatment of one of these conditions may vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 10 mg/kg, and preferably from about 0.001 mg/kg to about 10 mg/kg body weight per day. A unit dosage may contain from about 0.05 mg to about 500 mg of active ingredient, and may be administered one or more times per day. The daily dosage for administration by injection, including intravenous, intramuscular, subcutaneous, and parenteral injections, and use of infusion techniques may be from about 0.0001 mg/kg to about 10 mg/kg. The transdermal concentration may be that required to maintain a daily dose of from 0.0001 mg/kg to 10 mg/kg.

The specific initial and continuing dosage regimen for each patient will vary according to the nature and severity of the condition as determined by the attending diagnostician, the activity of the specific antisense oligonucleotide employed, the age of the patient, the diet of the patient, time of administration, route of administration, rate of excretion of the drug, drug combinations, and the like. The desired mode of treatment and number of doses of an antisense oligonucleotide of the present disclosure may be ascertained by those skilled in the art using conventional treatment tests.

Without being bound by any theory, it is believed that the use of the antisense oligonucleotides described herein increases a cancer cell's sensitization to other chemotherapeutic agents or immunotherapies. In another theory, it is believed that the use of the antisense oligonucleotides in combination with one or more chemotherapeutic agents, targeted inhibitors, or immunotherapies results in synergy. Thus, another aspect described herein is a method of treating cancer comprising administering the pharmaceutical compositions described herein, wherein the composition may be administered in combination with one or more chemotherapeutic agents, targeted inhibitors, immune checkpoint inhibitors, cell therapies, monoclonal antibodies, oncolytic virus therapies, cancer vaccines, or immune system modulators, including but not limited to the full spectrum of compositions and compounds which are known to be active in killing and/or inhibiting the growth of cancer cells.

Chemotherapeutic agents, may include, but are not limited to DNA-interactive agents, antimetabolites, tubulin interactive agents, anti-hormonals, anti-virals, ODC inhibitors and other cytotoxics such as hydroxy-urea. Any of these agents are suitable for use in the methods of the present invention. Chemotherapeutic agents further include, but not limited to cisplatin, carboplatin, camptothecin, indolizino, irinotecan, diflomotecan, exatecan, gimatecan, irinotecan, karenitecin, lurtorecan, rubitecan, silatecan, topotecan

Antibodies may be polyclonal or monoclonal antibodies, humanized or human, that bind to an epitope on any of the MUC1-C sequences described herein. Any suitable antibody targeting the MUC1-C is contemplated herein. In one aspect is a method of treating cancer comprising administering to a subject in need thereof, an effective amount of a pharmaceutical composition comprising any of the antisense oligonucleotides described herein in combination with one or more antibody. In another aspect, the antibody may be an anti-MUC1 antibody. In yet another aspect, the antibody comprises 3D1 or 7B8.

Targeted inhibitors comprise any targeted therapy, including but not limited to, therapies that target a specific gene or protein. These may include targeted therapies specific to a type of cancer. Examples of targeted inhibitors include inhibitors of HER2. BCR-ABL, EGFR, and VEGF, PARP or kinase inhibitors.

In another aspect, the antisense oligonucleotides of the present disclosure may be administered in combination with one or more additional therapeutic agent to improve the efficacy of other drugs. Potential other drugs include but not limited to: chemotherapeutic drugs including but not limited to camptothecin, indolizino, irinotecan, diflomotecan, exatecan, gimatecan, irinotecan, karenitecin, lurtorecan, rubitecan, silatecan, topotecan; targeted inhibitors; and antibodies.

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes).

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

### EXAMPLES

The antisense oligonucleotides described in the present disclosure may be conveniently and routinely made through any well-known technique of synthetic oligonucleotide synthesis, including solid phase synthesis and other methods known in the art.

Experiments in which MUC1-C was silenced in MCC26 MCCN cells were performed. It was found that MCC26 cells are dependent on MUC1-C for proliferation and survival.

### Example 1

Cell culture. MCC13 and MCC26 and UISO MCC cells were obtained and cultured in RPMI 1640 media (Corning®, Corning, NY, USA) supplemented with 10% FBS and 2 mM glutamine. Cells were cultured for 3-4 months. Authentication of the cells was performed by short tandem repeat (STR) analysis. Cells were monitored for mycoplasma contamination using the MycoAlert Mycoplasma Detection Kit (Lonza, Rockland, ME, USA).

Gene silencing and rescue. MUC1shRNA (MISSION shRNA TRCN0000122938; Sigma) or a control scrambled shRNA (CshRNA; Sigma) was inserted into the pLKO-tet-puro vector (Plasmid #21915; Addgene, Cambridge, MA, USA) by methods known in the art. MYCLshRNA was inserted into pLKO-tet-puro vector. MUC1-C or Flag-tagged MUC1-CD was inserted into plnducer20 (Plasmid #44012, Addgene). MYCL was inserted into the empty control pLenti CMV Blast DEST (706-1) vector (Plasmid #17451, Addgene). Cells transduced with the vectors were selected for growth in 1-4 μg/ml puromycin, 400-1000 μg/ml hygromycin, or 10 μg/ml blasticidin. Cells were (i) treated with 0.1% DMSO as the vehicle control or 500 ng/ml doxycycline (DOX; Millipore Sigma) and (ii) transfected with a MUC1/ASO (LG00788741; Qiagen, Hilden, Germany) or a control C/ASO (LG00000001; Qiagen) in the presence of Lipofectamine 3000 Reagent (Thermo Fisher Scientific, Waltham, MA, USA).

Analysis of MUC1 expression was performed using RNA-seq datasets derived from 23 MCC tumors and from 55 MCCP and MCCN tumors. Lysates from MCC26 cells transfected with 30 nM of the antisense oligonucleotides of SEQ ID NOs: 3-6 or a control antisense oligonucleotide (negative control B antisense LNA GapmeR) for 24 hours and subsequently were analyzed for MUC1-C mRNA levels by routine qRT-PCR using the following primer:

| Primer | FWD | REV |
|---|---|---|
| MUC1-C | AGACGTCAGC | GCCAAGGCAA |
| | GTGAGTGATG | TGAGATAGAC |

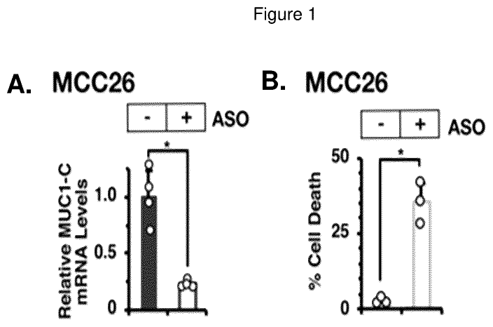

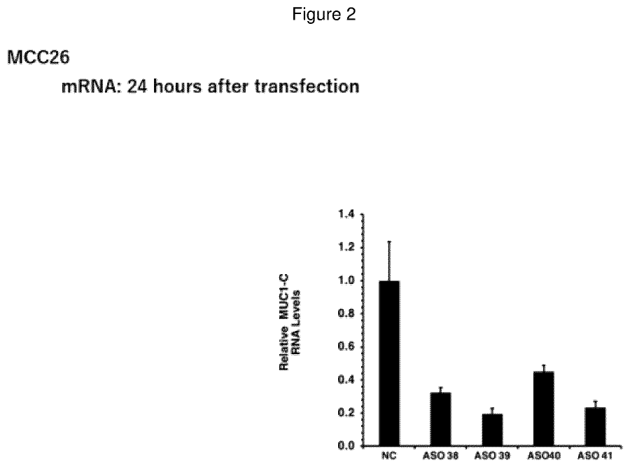

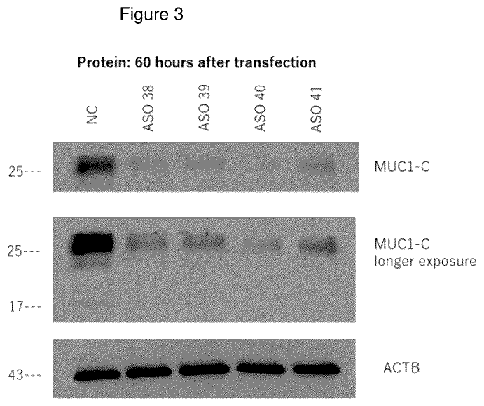

FIG. 1B and FIG. 2 describe results (mean±SD of three determinations) expressed as relative mRNA levels compared to that obtained for control transfected cells (assigned a value of 1).

### Example 2

In another experiment, MCC26 cells transfected with 30 nM of the antisense oligonucleotides of SEQ ID NOs: 3-6 or control antisense (negative control B antisense LNA GapmeR) oligonucleotide for 72 hours were monitored for cell death by trypan blue staining. FIG. 1C describes the results expressed as the % cell death (mean±SD of three separate determinations).

### Example 3

Immunoblot analysis. Immunoblotting was performed according to methods known in the art. Total lysates prepared from subconfluent cells were subjected to immunoblot analysis using anti-MUC1-C(HM-1630-P1ABX, 1:1000 dilution; Thermo Fisher Scientific).

### Example 4

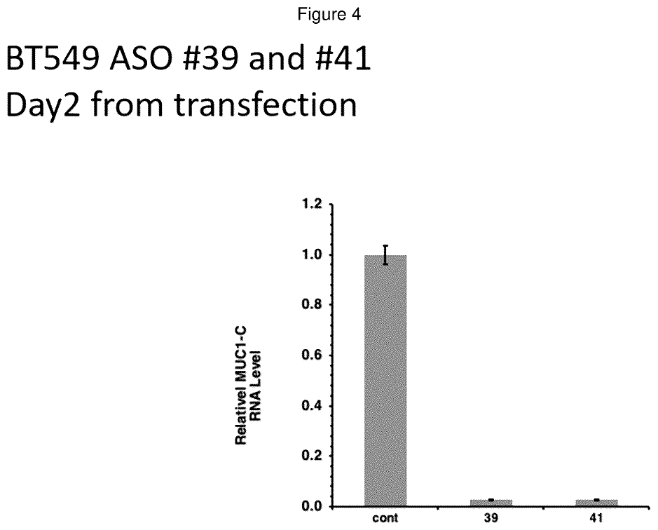

Further experiments were performed in which MUC1-C was silenced in BT-549 breast cancer cells using the antisense oligonucleotides of SEQ ID NOs: 5 and 6. All experiments were performed as described above. FIG. 4 describes the relative mRNA levels using qRT-PCR and the primers described above.

### Example 5

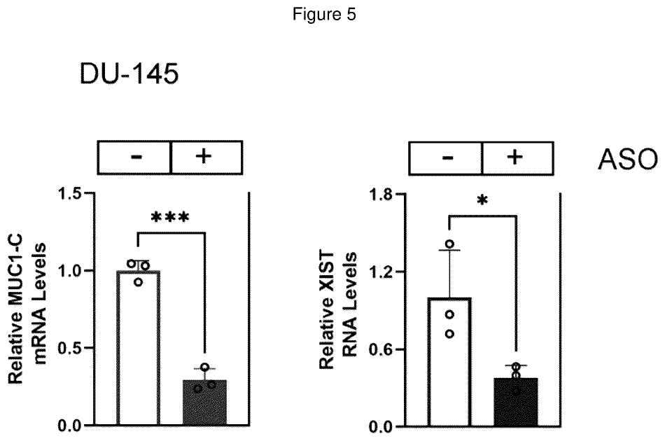

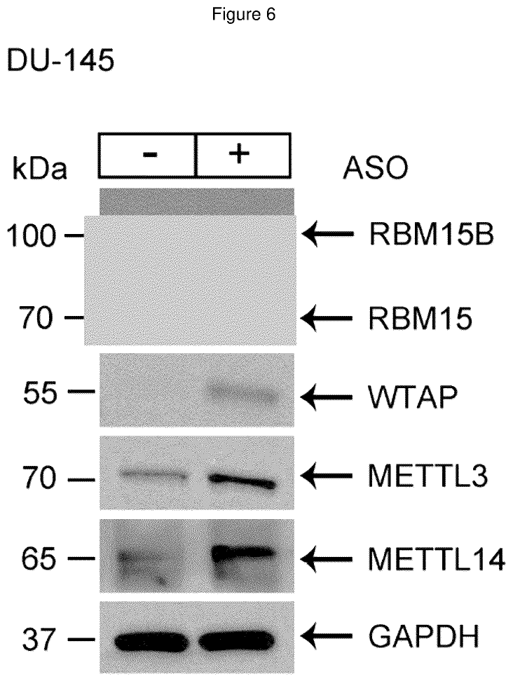

The effects of MUC1-C silencing were also studied in DU-145 prostate cancer cells using the antisense oligonucleotides of SEQ ID NO: 6 to understand potential upregulation or downregulation of genes, such as XIST. XIST is a non-coding RNA gene responsible for inactivation of the X chromosome in early embryonic development, and is often overexpressed in cancer cells. All experiments were performed as described above. Cell were treated with vehicle or non-antisense oligonucleotide control and relative mRNA levels were determined using qRT-PCR. FIG. 5 describes the relative MUC1-C mRNA levels and relative XIST RNA levels, where treatment with the antisense oligonucleotides of SEQ ID NO. 6 exhibited suppressed relative XIST mRNA levels. Lysates from the cells were obtained and immunoblotted with antibodies against the various proteins. FIG. 6 demonstrates ASO treatment results in upregulation of various pathway proteins.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

1. An antisense oligonucleotide of Formula (I):
AZₘGYnGXT
wherein
Z is independently in each occurrence any nucleotide;
Y is independently in each occurrence any nucleotide;
X is A or G;
m=3; and
n=7; or
a pharmaceutically acceptable salt thereof.

2. The antisense oligonucleotide of claim 1 comprising a length of 10-20 nucleotides.

3. The antisense oligonucleotide of claim 1, wherein the antisense oligonucleotide is a nucleotide sequence of SEQ ID NOs: 3-6.

4. The antisense oligonucleotides of claim 1, wherein the antisense oligonucleotide is optionally modified.

5. The antisense oligonucleotide of claim 1, wherein the antisense oligonucleotide target comprises an mRNA transcript.

6. The antisense oligonucleotide of claim 5, wherein the mRNA transcript is a MUC1-C mRNA transcript.

7. A pharmaceutical composition comprising one or more of the antisense oligonucleotide of claim 1 or a pharmaceutically acceptable salt thereof.

8. The composition of claim 7, further comprising a lipid nanoparticle.

9. A method of reducing cell viability comprising contacting a MUC1-C expressing cancer cell with the antisense oligonucleotides or the pharmaceutical compositions of claim 1.

10. A method of treating cancer comprising administering to a subject in need thereof, an effective amount of the composition claim 7.

11. The method of claim 10, wherein the cancer comprises a neuroendocrine cancer; breast cancer, colorectal cancer or prostate cancer.

12. The method of claim 11, wherein the neuroendocrine cancer is Merkel cell carcinoma.

13. The method of claim 10 wherein the composition may be administered intravenously or topically.

14. The method of claim 13, wherein the composition may be administered intravenously.

15. The method of claim 10, wherein the composition may be administered in combination with chemotherapeutic agents, targeted inhibitors, immunotherapies or immune checkpoint inhibitors.

16. The method of claim 15, wherein the immunotherapy is an anti-MUC1-C antibody.

17. A method of inhibiting survival of a cell, comprising contacting a cell that expresses MUC1-C with any of the antisense oligonucleotides or pharmaceutical compositions of claim 1.

18. A method of treating cancer comprising administering to a subject in need thereof, an effective amount of a pharmaceutical composition comprising an antisense oligonucleotide of Formula (II):
GPZₘGAYATXGA
wherein
P is G or T;
Z is independently in each occurrence any nucleotide;
Y is G or C;
X is A or T; and
m=3; or
or a pharmaceutically acceptable salt thereof.


SEQUENCE LISTING (6 sequences)

SEQ ID NO: 1 - DNA, 477 bp, Homo sapiens
  source 1..477: mol_type = genomic DNA
tctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtag

SEQ ID NO: 2 - protein, 158 aa, Homo sapiens
SVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 3 - DNA, 16 bp, Homo sapiens
  source 1..16: mol_type = other DNA
gtcgtggacattgatg

SEQ ID NO: 4 - DNA, 16 bp, Homo sapiens
  source 1..16: mol_type = other DNA
ggcaatgagatagaca

SEQ ID NO: 5 - DNA, 16 bp, Homo sapiens
  source 1..16: mol_type = other DNA
tactcgctcataggat

SEQ ID NO: 6 - DNA, 16 bp, Homo sapiens
  source 1..16: mol_type = other DNA
catgggtgtggtaggt